(19) United States
(12) Patent Application Publication
(10) Pub. No.: US 2026/0331239 A1
(43) Pub. Date: Oct. 22, 2026

(54) SYSTEMS AND METHODS FOR HEALTHCARE MANAGEMENT

(71) Applicant: Bradford James Roelofs, Ladera Ranch, CA (US)

(72) Inventor: Bradford James Roelofs, Ladera Ranch, CA (US)

(21) Appl. No.: 19/561,421

(22) Filed: Mar. 9, 2026

Related U.S. Application Data
(60) Provisional application No. 63/768,759, filed on Mar. 7, 2025.
(60) Provisional application No. 63/807,170, filed on May 16, 2025.

(51) Int. Cl. G16H 10/60; G16H 40/67

(52) U.S. Cl. CPC G16H 10/60; G16H 40/67

(57) ABSTRACT

Systems and methods for managing a person's healthcare are provided. Such systems and methods include placing and receiving a request at a server. They involve receiving a person's healthcare and contact information from a client system based on the request. A client identifier, free of protected health information, is assigned at the server. The server sends the client identifier and an HTML document with a care plan and submit button to the client system. The client system stores the identifiers, displays the HTML document, and sends an instruction to process the care plan when the submit button is selected. The server receives the instruction, combines the person's healthcare information with the client identifier, and generates an order to process the care plan according to the person's health information, enabling the person to order the care plan by selecting the submit button.

This application claims the benefit of U.S. Provisional Patent Application No. 63/807,170, filed May 16, 2025, and U.S. Provisional Patent Application No. 63/768,759, filed Mar. 7, 2025. The entire disclosures of the foregoing applications are incorporated herein by reference.

### TECHNICAL FIELD

The disclosure relates generally to the field of healthcare, specifically and not by way of limitation, some embodiments are related to healthcare management.

### BACKGROUND

Electronic health record (EHR) systems are widely used to store patient healthcare data, including diagnosis codes, procedure codes, medication information, laboratory values, encounter histories, demographic information, scheduling information, and other clinically relevant data. Although such systems contain large volumes of structured healthcare information, many EHR systems primarily function as repositories for storing and retrieving records and as workflow tools internal to provider organizations.

As a result, healthcare data stored in EHR systems is often underutilized for proactive patient engagement and care coordination. Patients frequently miss follow-up appointments, delay recommended diagnostic testing, fail to obtain recommended monitoring devices, or otherwise do not complete the next action associated with a diagnosed medical condition. These gaps in follow-through can increase healthcare costs, worsen clinical outcomes, and increase administrative burden on healthcare providers.

Existing reminder or outreach systems are generally limited to basic appointment reminders, portal messages, or staff-driven communications. Such systems typically do not continuously retrieve and interpret diagnosis-related clinical data from multiple electronic health record systems, and they do not convert stored clinical information into automated, diagnosis-relevant care-management workflows that guide patient engagement outside the provider's internal EHR environment.

Accordingly, there is a need for improved systems capable of retrieving structured healthcare data from one or more electronic health record systems, processing that data using external computing systems, identifying care opportunities associated with patient conditions, and generating automated patient engagement workflows and related healthcare actions based on the processed healthcare information.

### SUMMARY

Some systems and methods described herein may relate to automatic lifetime diagnosis codes and healthcare management.

One aspect includes systems and methods for managing a person's healthcare. The method includes placing a request and receiving the request at a server. Additionally, the method includes receiving a person's healthcare information at the server and contact information from a client system based on receiving the request. The method also includes assigning a client identifier to a person at the server, the client identifier absent any protected health information. Additionally, the method includes sending from the server to the client system the assigned client identifier and a Hypertext Markup Language (HTML) document identifying a care plan and including a submit button. The method also includes receiving and storing the assigned client identifier and receiving and displaying the HTML document at the client system. The method includes sending to the server an instruction to process the identified care plan by the client system in response to a selection of the submit button. Additionally, the methods includes receiving the request and combining the person's healthcare information associated with the client identifier of the client system to generate an order to process the care plan in accordance with the person's health information, whereby the person effects the ordering of the care plan by selection of the submit button by the server.

The features and advantages described in the specification are not all-inclusive. In particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the disclosed subject matter.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a diagram illustrating systems and methods for automated healthcare management.

FIGS. 2A-2B form a flow diagram illustrating an example method in accordance with the systems and methods described herein.

FIG. 3 illustrates a block diagram of a device for managing a person's healthcare.

FIG. 4 is a flowchart of an example process 400.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts.

Several aspects of telecommunication systems will now be presented with reference to various apparatus and methods. These apparatus and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, or other similar constructs, whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that a computer can access. By way of example, and not limitation, such computer-readable media can comprise random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer-executable code in the form of instructions or data structures that a computer can access.

As described above, it may be advantageous to use technology to mobilize the world's stored healthcare information and connect the stored healthcare to patient-centric solutions driven by the patient's personal health information. Some example embodiments may proactively engage and empower patients to put the patient's entire healthcare on autopilot. Autopilot may include but is not limited to a medical condition-driven healthcare management platform that may be customizable, wherein healthcare decisions may be driven by the patients needing care based on diagnosis codes. For example, autopilot may rely on the patient's medical codes, such as ICD codes, e.g., the 10th revision of the ICD (ICD-10), e.g., medical conditions, symptoms, or other codable items. ICD-10 Clinical Modification (ICD-10-CM) may be defined as, in all healthcare settings, a standardized and internationally recognized system of diagnosis codes that represent conditions and diseases, related health problems, abnormal findings, signs and symptoms, injuries, external causes of injuries and diseases, and social circumstances.

In some embodiments, healthcare information obtained from one or more electronic health record (EHR) systems is retrieved and processed by an external computing platform operating outside the native EHR environment. The external platform activates the retrieved healthcare data to generate automated workflows, notifications, and care-management actions that are executed independently of the EHR system.

In some example embodiments, the described system may leverage advanced data processing algorithms and secure communication protocols to enhance the efficiency and effectiveness of healthcare management. By utilizing machine learning models and predictive analytics, the system can provide personalized healthcare recommendations that adapt in real-time to the patient's changing conditions. This technological innovation addresses the limitations of manual and fragmented healthcare management by automating complex processes and ensuring data privacy and security.

In some embodiments, the systems described herein may be implemented as a computer-implemented healthcare activation platform configured to retrieve structured healthcare data from one or more electronic health record (EHR) systems. The platform may process and analyze the retrieved healthcare data outside the native electronic health record environment and may generate automated patient engagement actions and healthcare management workflows based on the analyzed data.

In some embodiments, the platform may generate patient-specific workflows based on structured healthcare information including diagnosis classifications, laboratory values, scheduling data, medication data, physiological monitoring data, and other healthcare data elements. Such workflows may include appointment reminders, scheduling prompts, treatment-adherence prompts, monitoring-device recommendations, educational content delivery, triage prompts, follow-up referrals, or care-program enrollment actions.

In some embodiments, the platform may form diagnosis-related cohorts using de-identified healthcare information and may associate healthcare products, services, educational resources, or other condition-relevant information with the cohorts. In some implementations, messages transmitted to patients may include recommendations or information associated with the patient's diagnosed condition while preventing disclosure of patient-identifying data to third-party providers of such products or services.

In some embodiments, the platform may record engagement outcomes associated with patient communications and may utilize the outcomes to improve or optimize future communications, recommendations, or workflows. The platform may operate across multiple healthcare providers, health systems, insurers, employers, government healthcare programs, or combinations thereof.

In some example embodiments, the system employs a specialized algorithm for processing healthcare requests, which includes data encryption and secure transmission protocols to safeguard patient information. The server's non-transitory computer-readable medium stores executable instructions that facilitate the dynamic generation of care plans based on the analyzed healthcare data. This approach not only automates the management of healthcare information but also provides a secure and efficient means of handling sensitive patient data.

An example system may load patient ID information and the patient's assigned ICD-10 codes into a computing platform. Some examples may provide the patient with a “1-Click” option to submit the information. For example, suppose the information is available on a computing device. In that case, the patient may have the option to open an application and the data and then submit that data using the application by clicking on a single button within the application, e.g., after opening the data file. Some examples may connect a single data file to a single application running on a device generally used by that single user. An automatic system of text, email, call reminders, or some combination of these, e.g., which may be specific to the standard of care (SOC) may be used to manage care for medical conditions for each patient, which drives the entire healthcare experience. In other words, the healthcare management function may be automated for the patient. In an aspect, patient information may be actively monitored to determine possible diagnosis of problems earlier in developing the underlying diseases. The world's health systems are currently reactive, e.g., based on potential patients complaining about various symptoms. Problems are dealt with after the symptoms occur.

In some embodiments, the healthcare information processed by the system may include structured healthcare data such as diagnostic codes, procedure codes, laboratory results, vital sign measurements, medication lists, and other structured clinical data elements obtained from electronic health record systems, wearable devices, or patient-reported inputs.

In some example embodiments, the system may have the ability to integrate disparate data sources, including electronic health records (EHRs), wearable devices, and patient-reported outcomes, into a cohesive and actionable format. The system's architecture includes specialized modules for data normalization, anomaly detection, and real-time alerts, which significantly enhance the ability to monitor and manage chronic conditions proactively.

In some embodiments, the system may generate automated patient engagement workflows based on the structured healthcare data. These workflows may include scheduling appointments, transmitting reminders, initiating monitoring requests, delivering educational content, and coordinating follow-up care actions associated with one or more identified medical conditions.

Some example embodiments enable the automatic suggestion of vital signs that may need to be monitored based on a patient's ICD-10 codes. For example, a patient with the high blood pressure code I10 may receive a suggestion to monitor the patient's blood pressure, which may be managed, e.g., through an application or by visiting a website via a provided link. In some embodiments, any suggestions the system makes may be sent to the application, e.g., as a link text to the patient, guiding the patient to a website for further action.

In some embodiments, healthcare services or products made available through the system may be diagnosis-driven, meaning that the services or products are automatically selected based on diagnostic codes or other clinical indicators associated with the patient's healthcare information.

In an example embodiment, systems and methods for managing a person's healthcare via the Internet may be provided. A request may be placed or received from a patient, authorized caregiver, healthcare provider, or electronic health record file at a client system and received by a server system. Based on the request, the electronic health record file may be reviewed. The review may be generated automatically, e.g., based on the electronic health record file. For example, a computing system implementing the systems and methods described herein may scan all electronic health record files within the computing system's files to determine the likelihood that someone has a medical condition requiring treatment and, more specifically, utilizing artificial intelligence (AI) to perpetually analyze the patient's need for care according to the combination of existing and new data entering the patient's stored healthcare information.

In an example embodiment, a server system may receive a person's healthcare information and contact information from a client system. The server system may then assign client identifier to a person. The client identifier may not include any protected health information in some aspects. The server system may then send the assigned client identifier to the client system. The server system may also send an HTML document identifying the care plan. The HTML document may include a submit button to allow, e.g., the client to submit additional information within the HTML document or indicate that the patient or client wishes to proceed with the proposed care plan.

Some example embodiments may integrate private industry into the EHR ecosystems, allowing healthcare products and services that improve patients' medical conditions to be directly offered through personalized 1-click purchase links ordered by their medical providers. For example, a health system with 1 million members may have 400,000 members with hypertension. In some embodiments, a matching engine may automatically send out 400,000 1-click purchase links for blood pressure monitors to members with hypertension by the members' cellphones, e.g., in the form of a text message, in-app message, or both. For example, these messages may be from their doctor. With each purchase, patients may earn rewards and money to pay coin balances and get discounted premiums with that provider, increasing revenue, patient compliance, healthcare outcomes, financial relief for healthcare systems, or some combination of these.

In some example embodiments, the client system may be designed with an intuitive user interface that may allow patients and healthcare providers to interact seamlessly with the healthcare management platform. The HTML document transmitted to the client system includes interactive elements, such as dynamic forms and real-time validation, which streamline the process of updating and submitting healthcare information. This user-centric design may improve patient engagement and compliance with prescribed care plans.

In some embodiments, the system may dynamically generate care-management actions based on changes to the patient's healthcare data, including the addition of new diagnosis codes, updates to vital sign measurements, or newly received laboratory results.

The client system may receive and store the assigned client identifier. The client system may also receive and display the HTML document. In response to the selection of the submit button, the client system may send a request to the server system to process the identified care plan. The server system may receive the request and combine the person's information associated with the client identifier of the client system to generate an order to process the care plan. The care plan may be processed according to the person's health information whereby the person effects the ordering of the care plan by selecting the submit button.

In some embodiments, patient engagement actions generated by the system may include condition-specific communications delivered through one or more channels including text message, email, in-application notifications, or automated telephone calls.

In an example embodiment, a server system may receive a person's healthcare information, including symptoms, medical conditions, diagnoses, and ICD-10 codes. ICD-10 is the tenth revision of the International Statistical Classification of Diseases and Related Health Problems (ICD), a medical classification list by the World Health Organization (WHO). ICD-10 contains codes for diseases, signs and symptoms, abnormal findings, complaints, social circumstances, and external causes of injury or diseases.

In an example embodiment, ICD-10 codes may include the person's health codes and contact information from the client system. The server system may assign a client identifier to the person absent from protected health information. The server system may also associate the assigned client identifier with the recipient's healthcare information, e.g., health codes such as ICD-10. Each health code may be assigned a client health code identifier. The server system may send the assigned client identifier to the client system. The server system may also send an HTML document identifying the following for each client health code identifier to the client system and including a submit button: a schedule of healthcare appointments, targeted educational content, assignment of private chat rooms and groups, and a care plan a schedule of healthcare reminders” that may be sent by one or more of text message, email, and telephone call, or other form of communication.

In an example, the ICD-10 codes may map to descriptions of the medical conditions relating to the codes. The ICD-10 codes may form the basis of a database that specifically attaches the frequency of office visits to care for that ICD-10 code related medical condition properly. Accordingly, using AI, a system may automatically change the frequency of office visits and a care plan as medical conditions are resolved, or new medical conditions are added. Additionally, the systems and methods described herein may have the ability to allow a medical provider to click on a button to customize the frequency of care and testing upon receiving the automatic notification from the software that a patient's vital sign reading is out of normal parameters.

In an example embodiment, the system may utilize machine learning models to analyze the patient's healthcare data and provide predictive insights into potential health risks. These models may be trained on large datasets of anonymized healthcare records to ensure accuracy and reliability. By predicting potential complications before they arise, the system may enable proactive intervention and personalized treatment plans that can significantly improve patient outcomes.

Some embodiments may use in-application patient compliance for vital signs monitoring, AI-driven appointment scheduling, and patient education may be rewarded with points and cash for coinsurance balances or discounted premiums. An example system may source data from various medical records, including, but not limited to, one or more EHRs, ICD codes, past and present appointments, patient-entered symptoms, descriptions of conditions, and information from healthcare providers, caregivers, insurance carriers, or employers.

The client system may receive and store the assigned client identifier and associated health code identifiers. Additionally, the client system may receive and display the HTML document. In an example, the HTML document may include an automated care plan. For example, a system may provide a plan for care and a schedule of reminders to initiate care appointments and testing necessary to care for the ICD-10 code/condition. In response to the selection of the submit button, the client system may send to the server system a request to schedule displayed appointments, educational content, private chat rooms and groups, and the care plan options associated with each client's health code identifier. Again, some examples of embodiment may include the automatic attachment of reminder text, reminder email, and/or reminder calls that may be scheduled according to the standard of care for that condition or condition. In some example embodiments, 1-Click Access to medical records may be provided. For example, a patient may click a button. A request may be sent to the medical records department of an electronic health records (EHR) company or provider office with a notice stating by law that the EHR company or provider has to comply within 15 days “in any format” preferred by the patient request, for example (or some other medical records providing requirement based on laws, contracts between the parties, or some other requirements or agreements). A patient may also have the ability to add other healthcare providers, such as the patient's contact information, allowing the system to contact that provider and provide the patient the ability to request a complete copy of the patient's medical records. In an example, a goal may be to provide 1-Click access to a centralized medical record from the patient's healthcare providers, e.g., all the patient's healthcare providers or, in some cases, some subset of the patient's healthcare providers.

In some embodiments, automated appointment scheduling, reminders, or both may be driven by standard-of-care templates and customized by healthcare providers. For example, patients with hypertension may receive automated reminders for appointments, e.g., every three months, e.g., until the condition is resolved.

The server system may receive the requests and combine the person's healthcare information associated with the client identifier and client health codes to generate an order to manage the person's healthcare proactively. Additionally, the server system may utilize automated scheduling, permission-based text messages, email reminders, and telephone calls, per the healthcare information, demographic, and contact information, whereby the person affects the ordering of the automated healthcare schedule and plan by selecting the submit button.

In an example embodiment, the automated scheduling and reminder system may be driven by a sophisticated rules engine that may take into account the patient's medical history, current health status, and standard-of-care guidelines. This engine can dynamically adjust appointment frequencies and notification schedules based on real-time data, ensuring that patients receive timely and appropriate care. This level of automation and personalization may be a significant advancement over traditional manual scheduling methods.

In an example embodiment, a patient may visit a website (including applications or “apps”) and enter conditions, cellphone number, email, or some combination of these. The patient may then click a disclosure for approval and “submit” the entered information. A response based on the submission may then state something like, “Great Job, You Now Have Control of Your Own Healthcare!” “An automatic link has been sent to customize care,”—from which a system may gather the remaining demographic and healthcare information with all disclosures legally necessary. Once fully active, the dashboard may display a choice of healthcare providers with appointments set, a statement indicating automatic text/email, telephone calls, or both may be sent as reminders, a list of Social Media Groups with the same conditions, and links to connect or join, list types of condition-specific educational content that may be provided, all with the option to opt-in, out, change provider selected, and similar actions. Accordingly, an example embodiment may provide an automated health provider scheduling solution driven by the patient's conditions and standard care plan regarding the frequency and testing required to manage it.

In an example, reminders may currently be done in mass, like, for example, your dentist appointment, but loaded in bulk and not driven by ICD-10 automatically. While the systems and methods described herein are discussed with respect to healthcare, it will be understood that the systems and methods described herein may be applied to other areas. For example, home care, assisted living, concierge medicine, behavioral medicine, education, veterinary medicine, and finance as it relates to weaknesses in financial statements, accounts, and goals.

In some embodiments, the system may also include an automated marketplace for healthcare products and services personalized to each patient's ICD codes and other healthcare information from the patient's medical record. These products and services may be sent to patients'cellphones, emails, other formats such as 1-click purchase links, or some combination.

In some embodiments, the system may generate targeted recommendations for healthcare products or services associated with a patient's diagnostic codes or healthcare data, including medical devices, monitoring equipment, educational resources, or other condition-related services.

The systems and methods described herein may be applied outside the medical context. For example, in some embodiments, a grading system may be applied analogously to the ICD-10 condition system in medicine that for finance may have equivalent descriptive codes assigned from which an automated system of reminders may be applied to enable persons to manage their finances more effectively and receive reminders before and after noted patterns in spending behaviors with the goal of for example, fore-warning that the person's normal behavior of an expensive restaurant on the weekend may not be a good idea based on recent scores and apply suggestive alternatives.

The systems and methods described herein may be applied to employers, for example, in an embodiment, a grading coding system for employers, or some combination of these may be used to track employee satisfaction, performance, and other factors that may be put into place the same automated platform to increase employee satisfaction, for example, employee frustration mounts, connects to vacation days balance and texts a suggested opportunity for time off or to schedule a meeting with superior.

In some example embodiments, the system may provide automated, personalized patient education, including medical condition education content, Really Simple Syndication (RSS) feeds, news, clinical trials, and breakthroughs sent to the patient's cellphone.

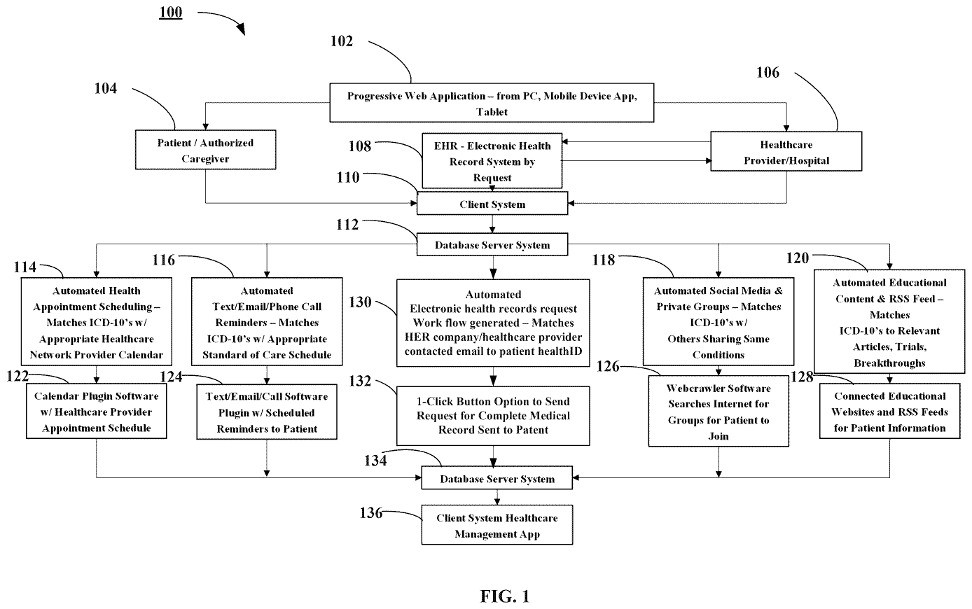

FIG. 1 is a diagram 100 illustrating systems and methods for automated healthcare management. Diagram 100 includes a progressive web application that may be run on a personal computer (PC), mobile device, tablet, or other computing device. The progressive web application may be executed by the patient or authorized caregiver in one example 104 or a health care provider or hospital in another example 106. The health care provider or hospital, for example, 106, may access electronic health care record systems by request 108. The patient or authorized caregiver 104 and the health care provider or hospital example 106 may use a client system 110. The client system 110 may be used to access a database server system 112. The database server system 112 may have access to automated health appointment scheduling, including matches to ICD-10 codes with an appropriate health care network provider calendar 114. The database server system 112 may also have access to automated text/email/phone call reminders that match ICD-10's with an appropriate standard of care schedule 116. Additionally, the database server system 112 may have access to automated social media and private groups that match ICD-10's with others sharing the same conditions 118. The database server system 112 may also have access to automated educational content and RSS feed that matches ICD-10's to relevant articles, trials, and breakthroughs 120.

An example embodiment may include the ability for patients to click and request a full copy of the patient's electronic medical record from the provider that prescribed a particular service and, in some embodiments, from other healthcare providers with the goal of providing one Centralized Electronic Medical Record and the ability to share with other providers via a secure link.

In some embodiments, the database server system may further include additional modules configured to perform healthcare engagement optimization and related services. Such modules may include messaging orchestration modules, care-gap identification modules, cohort-generation modules, data normalization modules, and outcome analysis modules configured to process structured healthcare data retrieved from one or more electronic health record systems.

In some embodiments, the system may further include monetization or incentive modules that associate diagnosis-relevant healthcare products, services, educational resources, or financial incentives with patient communications generated by the system. Such modules may include recommendation engines, healthcare product marketplaces, payer incentive engines, affiliate or referral systems, or other mechanisms configured to attach diagnosis-relevant services or offers to communications generated by the platform while maintaining appropriate privacy protections for patient-identifying information.

In some embodiments, the system may operate as an external healthcare activation platform configured to function outside one or more electronic health record (EHR) systems. Rather than requiring the electronic health record system itself to generate all patient engagement actions, the platform retrieves structured healthcare data through authorized interfaces, processes the retrieved data in a separate computing environment, and determines appropriate patient actions. This architecture enables the platform to operate across heterogeneous electronic health record systems and across multiple healthcare entities.

In some embodiments, the platform may include one or more components including a data-ingestion interface, a normalization engine, a clinical-classification processing engine, an event-trigger detection engine, a care-gap detection engine, a messaging orchestration engine, a service and recommendation engine, a diagnosis-driven advertising engine, an outcome optimization engine, and administrative, payer, provider, or population-health interfaces.

### Data Ingestion and EHR Integration

In some embodiments, the platform retrieves data from one or more electronic health record systems using application programming interfaces (APIs), flat-file transfers, Health Level Seven (HL7) interfaces, Fast Healthcare Interoperability Resources (FHIR) resources, batch exports, real-time event streams, secure database connections, or other authorized interoperability mechanisms. Retrieved data may include diagnosis codes, procedure codes, laboratory values, medication information, appointment schedules, care plans, demographic information, insurance data, remote monitoring values, provider information, and communication preferences.

In some embodiments, the platform retrieves newly added diagnosis codes or other changed patient data at scheduled intervals. In other embodiments, the platform receives event notifications when new clinical data is posted. In still other embodiments, the platform performs both periodic polling and event-driven ingestion. The platform may support integration with multiple electronic health record vendors simultaneously and may maintain source-specific adapters for each source system.

The ingestion layer may create a secure internal representation of source data, store source identifiers, maintain mappings between source records and internal records, and log ingestion events for traceability.

### Data Normalization

Because different electronic health record systems represent similar clinical information differently, the normalization engine converts heterogeneous source data into a common internal schema. Diagnosis classifications, problem-list entries, laboratory codes, medication records, appointment types, and provider references may be translated into standardized internal representations.

Normalization may include code mapping, deduplication, timestamp alignment, patient matching, provider matching, location matching, unit normalization, text extraction from structured fields, field validation, confidence scoring, and data completeness checks. By normalizing data across multiple sources, the platform may operate as a unified activation layer rather than as a tool limited to a single EHR instance.

### Clinical Classification Processing

The platform processes structured clinical classifications associated with patient conditions. In some embodiments these classifications include diagnosis codes. In broader embodiments, the platform may also use procedure codes, laboratory classifications, medication classes, device classes, risk scores, or other condition-indicating classifications.

The processing engine may associate one or more classifications with one or more standard-of-care schedules, risk pathways, educational resources, monitoring requirements, service options, or messaging templates. A single patient may have multiple concurrent conditions, and the engine may prioritize or combine actions across conditions based on severity, recency, comorbidity, provider preference, payer rules, or organizational rules.

### Event Trigger Detection

In some embodiments, the platform continuously or periodically detects events that trigger activation workflows. Triggering events may include the addition of a diagnosis code, abnormal laboratory results, missed appointments, overdue follow-up visits, medication initiation or discontinuation, discharge from a healthcare facility, new referrals, remote monitoring values, completion of services, non-response to prior messages, or expiration of a standard-of-care timer.

When an event occurs, the event-trigger engine may generate one or more workflow tasks. Such tasks may be patient-facing, provider-facing, administrative, or payer-facing. The engine may suppress duplicate triggers, rank triggers by urgency, bundle related actions, and defer actions based on communication windows or organizational rules.

### Care Gap Detection

The care-gap detection engine compares current patient records against one or more expected actions or schedules associated with the patient's condition profile. For example, where a diagnosis is associated with a recommended follow-up interval or recommended monitoring device, the engine may determine whether the patient is overdue, untreated, non-monitored, or otherwise outside the expected care pathway.

Care gaps may be defined by provider rules, payer rules, public-health protocols, organizational protocols, national standards, or machine-learned patterns. The platform may rank gaps by clinical urgency, cost impact, payer impact, likelihood of response, or probability of adverse outcome in order to prioritize engagement actions.

### Messaging and Engagement

The messaging orchestration engine may transmit communications through one or more channels including SMS messages, multimedia messages, email, push notifications, web interfaces, chat interfaces, voice systems, or combinations thereof. Communications may include reminders, scheduling prompts, patient education content, treatment-adherence prompts, requests for symptom input, prompts to record physiological measurements, links to provider-approved services, or confirmations of completed actions.

### Recommendation Engine

In some embodiments, the platform may recommend healthcare services, products, or programs relevant to a patient's diagnosis profile. Recommendations may include diagnostic testing, remote monitoring devices, telehealth programs, educational modules, provider follow-up services, wellness programs, medication adherence supports, or other healthcare-related services.

Recommendations may be generated using rule-based systems, statistical models, machine-learning models, or combinations thereof. In some embodiments, the recommendation engine generates one-click or low-friction action links allowing the patient to schedule, request, enroll in, or obtain a recommended service.

### Diagnosis-Driven Advertising

In some embodiments, the platform may include a diagnosis-driven advertising engine configured to associate diagnosis-relevant products or services with patient communications. The platform may generate de-identified cohorts of patients sharing one or more conditions, risk profiles, or engagement characteristics. Advertisers or service providers may target such cohorts without receiving patient-identifying data.

Advertising content may be appended to, embedded within, or otherwise associated with patient communications generated for care-management purposes. For example, a patient receiving a follow-up reminder associated with a cardiovascular condition may receive, within the same communication workflow, a diagnosis-relevant device, service, or educational offer.

### Outcome Optimization

The platform may measure message delivery, message engagement, completed scheduling actions, completed purchases, device activation, adherence events, appointment attendance, and downstream healthcare outcomes. These engagement signals may be used to optimize future communications, recommendation ranking, care-gap prioritization, or workflow sequencing.

### Multi-Entity Deployment

The platform may be deployed by provider practices, hospital systems, integrated delivery networks, insurers, self-funded employers, government healthcare programs, public-health entities, or combinations thereof. In some embodiments, multiple participating entities contribute data to a common activation platform while maintaining entity-specific governance and branding.

### Example Workflow

In an example workflow, a diagnosis classification added to a patient record within an electronic health record system may be retrieved by the ingestion interface. The normalization engine converts the source data into the platform schema. The event detection engine identifies the diagnosis addition as a triggering event. The care-gap engine determines that the patient is due for follow-up care or monitoring. The messaging engine then generates a communication to the patient containing a scheduling reminder and, in some embodiments, a diagnosis-relevant service recommendation.

### Healthcare Engagement System Architecture

In some embodiments, the disclosed systems may include a computer-implemented healthcare engagement system configured to retrieve structured healthcare data from one or more electronic health record systems and to generate automated patient engagement workflows based on the retrieved healthcare data. The system may include one or more server systems or processing systems executing software modules that retrieve data from electronic health record systems and analyze such data to determine appropriate patient engagement actions.

In some embodiments, the retrieved healthcare data may include diagnosis classification codes associated with patient conditions. For example, the healthcare data may include diagnosis codes stored within electronic health record systems that represent diseases, symptoms, medical conditions, injuries, or other clinically relevant classifications associated with a patient.

### Data Normalization

In some embodiments, the system may include a normalization engine configured to convert healthcare data originating from multiple electronic health record systems into a common internal schema. Because different electronic health record vendors often represent similar clinical information using different data formats, field names, or coding conventions, the normalization engine may translate heterogeneous data into standardized internal representations that can be processed by downstream workflow engines.

### Event Trigger Detection

In some embodiments, the system may include an event-trigger detection engine configured to detect changes in patient records and initiate automated engagement workflows in response to such changes. For example, the event-trigger detection engine may detect the addition of a new diagnosis classification, an abnormal laboratory value, an overdue appointment, a missed appointment, a medication event, a discharge event, or a remote monitoring event.

Upon detecting such an event, the system may generate one or more engagement workflows configured to prompt patient actions, notify healthcare providers, or initiate administrative actions associated with the detected event.

### Care Gap Detection

In some embodiments, the system may include a care-gap detection engine configured to determine whether a patient has failed to complete an expected healthcare action associated with a condition profile. For example, the system may determine that a patient has not completed a recommended follow-up appointment, diagnostic test, monitoring activity, or other healthcare action associated with a diagnosed condition.

### Automated Patient Engagement Workflows

The automated patient engagement workflows generated by the system may include a variety of patient communications and engagement actions. For example, the workflows may include appointment reminders, treatment adherence prompts, or delivery of educational health content associated with a patient's medical condition.

In some embodiments, patient communications may be transmitted through one or more messaging channels including short message service (SMS) messaging, electronic mail messages, push notifications, or other electronic communication mechanisms.

### Service Recommendation Engine

In some embodiments, the system may include a healthcare service recommendation engine configured to associate a healthcare service, device, or program with a patient condition profile. The recommendation engine may analyze diagnosis classifications, care gaps, or other healthcare data to determine which services or products may be relevant to a patient's condition.

In some implementations, the recommendation engine may generate low-friction action links allowing a patient to schedule, enroll in, request, or obtain a service or product directly from a communication generated by the system.

### External Operation Relative to EHR Systems

In some embodiments, the healthcare engagement system operates external to native electronic health record software workflows. Rather than relying on internal workflow tools within a single electronic health record system, the platform retrieves data from one or more electronic health record systems and generates engagement workflows using a separate computing environment.

### Outcome Optimization

In some embodiments, the system may include an outcome optimization engine configured to analyze engagement outcomes and refine later engagement workflows based on observed engagement results. Engagement outcomes may include message delivery confirmation, message opening events, click-through events, patient responses, appointment completion events, purchases, device activations, or treatment adherence events.

### Diagnosis-Driven Advertising

In some embodiments, the system may generate de-identified patient cohorts based on diagnosis classifications or condition profiles. Such cohorts may be used to associate healthcare products or services with groups of patients sharing similar conditions while avoiding disclosure of patient-identifying information.

In some implementations, advertisements or service recommendations may be delivered within automated patient communications. For example, a communication generated as part of a care-management workflow may include a diagnosis-relevant product, service, or educational offering associated with the patient's condition profile.

### Healthcare Activation Platform

In some embodiments, the platform may include an activation engine configured to generate automated care-management workflows using data retrieved from multiple electronic health record systems. The platform may further include a cohort engine configured to generate de-identified diagnosis-based patient cohorts and an advertising engine configured to associate diagnosis-relevant healthcare advertising with automated patient communications.

In some embodiments, the platform may include population-health interfaces configured to display aggregated care gaps, engagement metrics, diagnosis distributions, or other population-level healthcare indicators across multiple healthcare entities.

In some implementations, automated patient communications generated by the platform may include both a care-management component and a diagnosis-relevant service or advertising component.

The platform may retrieve data from multiple heterogeneous electronic health record systems using different integration mechanisms including application programming interfaces, data feeds, batch transfers, or event-driven interfaces.

In some embodiments, the platform may record monetization outcomes associated with diagnosis-driven advertising and may use the monetization outcomes to optimize later advertising selection or later engagement workflows.

Some example embodiments may automate electronic health records requests. For example, patients may send requests to the patient's provider's EHR system with a 1-click option, and, in some cases, by law, the EHR may have two weeks (or some other time frame in other areas) to comply with the patient's desired format.

As illustrated in FIG. 1, an automated electronic health records request may result in a workflow 130. The workflow may match a company, a healthcare provider, a contact email, or some combination of a patient's health identification. A 1-Click Button option may cause a request to be sent a request for a complete medical record to a patient 132.

The automated health appointment scheduling, including matches to ICD-10 codes with appropriate health care network provider calendar 114, may be coupled to the calendar plugin software with healthcare provider appointment schedule 122. The automated text/email/phone call reminders that match ICD-10's with an appropriate standard of care schedule 116 may be coupled to the text/email/call software plugin with scheduled reminders to patient 124. Additionally, the automated social media and private groups that match ICD-10's with others sharing the same conditions 118 may be coupled to the WebCrawler software search Internet for groups for patients to join 126. The automated educational content and RSS feed matches ICD-10's to relevant articles, trials, and breakthroughs 120, which may be coupled to the connected educational websites and RSS feeds for patient information 128. The blocks 122, 124, 126, 128 may be coupled to the database server system 134, which feeds the client system healthcare management application 136. The systems and methods described herein may take a number of manually performed functions and automate them through the IDC-10 codes (or any other medical codes that may be developed now, in the future, or that may have been previously developed.)

In some aspects, the automated healthcare management system described herein may take various manually performed functions and automate them using, e.g., ICD-10 codes or any other medical codes. An example embodiment may include receiving, by the company, all patient demographics, ICD-10 codes, vital signs, and other information from a physician, hospital, or other medical providers, allowed access to the patient's electronic health records. Once received, the data may be loaded in mass. The software may automatically process a request, send a text, email link, other communication, or some combination of these, to the patient who, after entering a code, e.g., a 6-digit code, may open the link to view the already populated data. The patient may have the ability to change the data. The patient may then click submit the data, at which time all features described herein may be processed.

Some example embodiments may allow patients to take and enter their own vital signs. In an example embodiment, any vital signs out of normal range may automatically generate an email to the patient's physician, who may prescribe any needed services. At this time, the physician may have the opportunity to customize the parameters or respond, causing an appointment request to be texted to the patient.

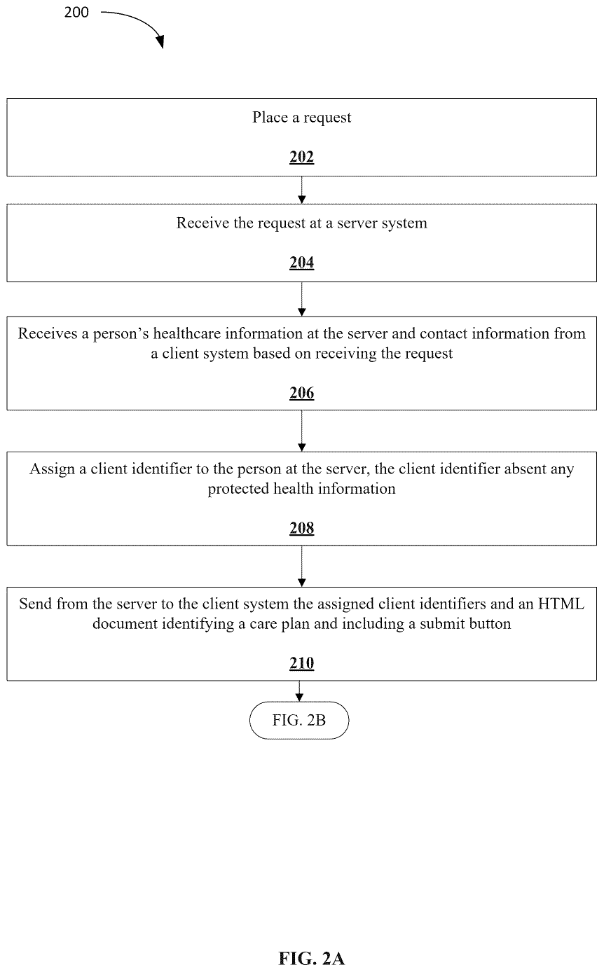

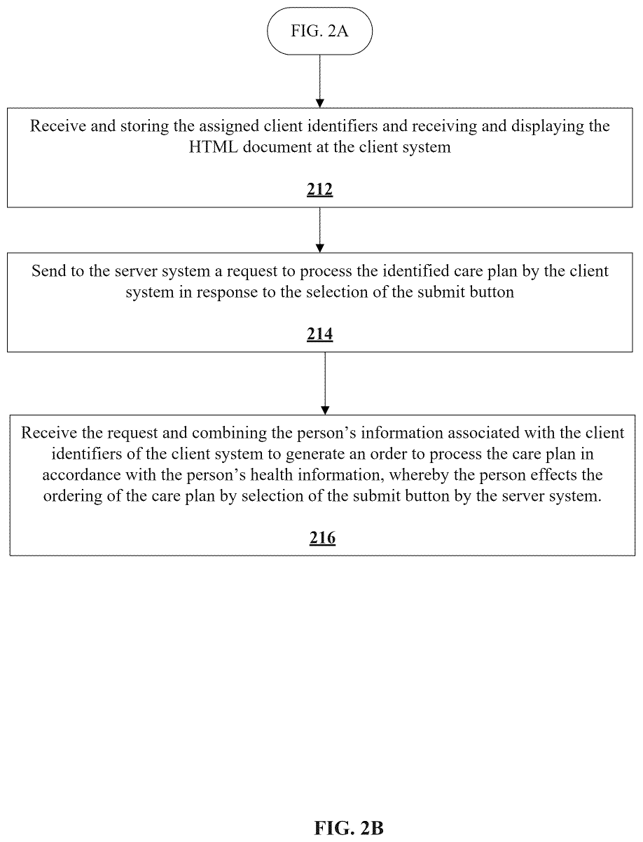

FIGS. 2A-2B form a flow diagram 200 illustrating an example method in accordance with the systems and methods described herein. (See FIG. 2A.) The method may be method 200 for managing a person's healthcare. The method 200 includes placing a request (202). Method 200 also includes receiving the request (204) from a server system. Additionally, method 200 includes receiving a person's healthcare information at the server and contact information from a client system based on receiving the request (206). Method 200 also includes assigning a client identifier to the person. In some example embodiments, the assignment may be performed at the server (208). The client identifier may, in some cases, not include any protected health information. Additionally, method 200 includes sending the assigned client identifier from the server to the client system, an HTML document identifying a care plan, and a submit button (210). (See FIG. 2B.) Method 200 also includes receiving and storing the assigned client identifier and receiving and displaying the HTML document at the client system (212). Additionally, method 200 also includes sending to the server system a request to process the identified care plan by the client system in response to the selection of the submit button (214). Method 200 also includes receiving the request and combining the person's information associated with the client identifier of the client system to generate an order to process the care plan in accordance with the person's health information, whereby the person affects the ordering of the care plan by selection of the submit button by the server system (216).

Placing a Request (202) may include placing a request via a client system configured to interface with a server. The client system may include a user interface that allows the user to input necessary information such as personal details, healthcare requirements, and other relevant data. The request may be initiated by the patient, an authorized caregiver, or a healthcare provider using the client system.

Once the request is placed, it is received by a server system. Receiving the Request (204) at the server may include handling multiple incoming requests, ensuring each request is logged and processed efficiently. This step may help ensure that the server is ready to perform further actions based on the received request.

Receiving Healthcare Information (206) may include extracting and analyzing a person's healthcare information and contact information provided in the request. The server uses specialized algorithms to securely handle and process this sensitive data. This step may involve one or more sub-steps such as validating the authenticity of the request, parsing the received data to identify key healthcare information, and storing the extracted information in a secure database for further processing.

Assigning a Client Identifier (208) to the person at the server may include a client identifier that is generated through a cryptographic process to ensure security and privacy. The identifier may be unique to each individual and does not contain any protected health information. Some embodiments may include one or more of generating a unique identifier using cryptographic algorithms, associating the identifier with the person's healthcare information in the database, and ensuring the identifier is stored securely and can be retrieved efficiently for future use.

Sending Assigned Client Identifier and HTML Document (210) may include the server transmitting the assigned client identifier along with an HTML document to the client system. The HTML document may identify a care plan and includes interactive elements such as a submit button for user input. This process may include one or more of creating an HTML document with the necessary interactive elements, embedding the assigned client identifier within the HTML document, and sending the HTML document securely to the client system via the network.

Receiving and Storing Assigned Client Identifier (212) may include the client system receiving and storing the assigned client identifier. The client system may also displays the HTML document to the user, allowing them to review and interact with the care plan. This step ensures that the client system is ready to process user inputs. This process may include one or more of storing the received client identifier in a local or secure storage system, rendering the HTML document on the client system's user interface, and preparing the system to handle user interactions with the HTML document.

Sending Request to Process Identified Care Plan (214) may occur upon user interaction with the HTML document, such as selecting the submit button. The client system may send an instruction to the server to process the identified care plan. This step may involve securely transmitting the user's decision back to the server for further action. For example, this process may include one or more of capturing the user's interaction with the HTML document, formatting the interaction data into a request for the server, and sending the request securely to the server for processing.

Receiving and Combining Information to Generate an Order (216) may involve the server receiving the request and combining the person's healthcare information associated with the client identifier to generate an order. The execution of the care plan may involve a machine-learning model to optimize treatment recommendations based on the person's health information. This step may help ensure that the person effects the ordering of the care plan through secure server-client communication. This process may include one or more of receiving the request from the client system, retrieving the person's healthcare information using the client identifier, utilizing a machine-learning model to analyze the healthcare data and optimize treatment recommendations, generating an order to process the care plan in accordance with the person's health information, and sending a confirmation of the processed care plan back to the client system.

These steps may collectively illustrate a comprehensive method for managing a person's healthcare, ensuring security, privacy, and efficient processing of healthcare information through advanced technological means.

An example embodiment may include receiving, by the company, all patient demographics, ICD-10, vital signs, and other information from a physician allowed access to the patient's electronic health records. Once received, the data may be loaded en mass. The software may automatically process a request to send a text and/or email link to the patient, who, after entering a code, e.g., a 6-digit code, may open the link to view the already populated data, may have the ability to change the data, and then may click submit at which time all features as described herein may be processed.

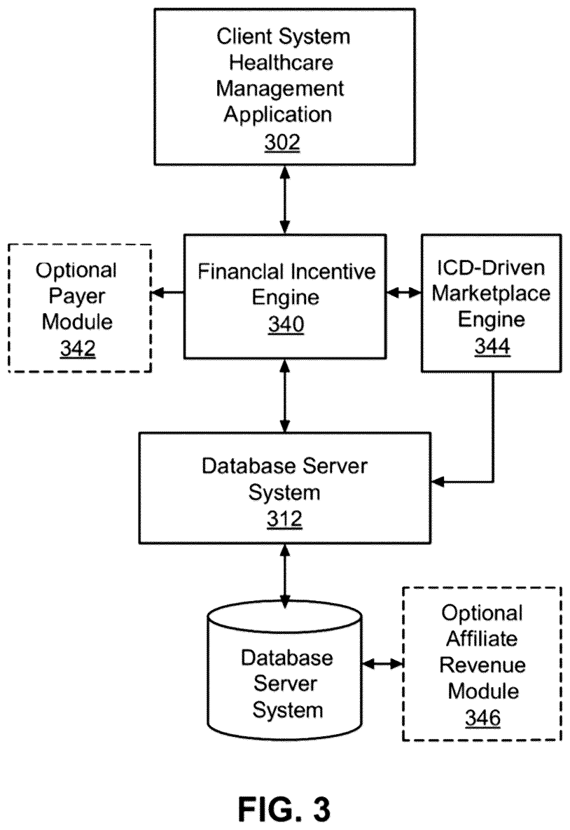

FIG. 3 illustrates a block diagram of a device 300 for managing a person's healthcare. The device 300 comprises one or more processors 302 configured to perform various functions and interact with a client system 304 and a server 306. The client system 304 is configured to interface with the server 306 via a network 308.

The processors 302 are configured to place a request via the client system 304. The server 306 includes a non-transitory computer-readable medium 310 storing executable instructions. Upon receiving the request at the server 306, the processors 302 execute a specialized algorithm 312 to extract and analyze the person's healthcare information and contact information from the client system 304. This specialized algorithm includes data encryption and secure transmission protocols to safeguard patient information, providing a significant technological improvement over traditional methods.

The processors 302 assign a unique client identifier to the person at the server 306, ensuring security and privacy through a cryptographic process 314. The server 306 then transmits the assigned client identifier and an HTML document 316 to the client system 304. The HTML document 316 identifies a care plan and includes interactive elements for user input, such as dynamic forms and real-time validation, which streamline the process of updating and submitting healthcare information. This user-centric design improves patient engagement and compliance with prescribed care plans.

The client system 304 receives and stores the assigned client identifier and displays the HTML document 316. The client system 304 is configured to interface with user input devices 318, allowing user interaction with the HTML document 316. Based on the user interaction, an instruction to process the identified care plan is sent back to the server 306.

The processors 302 at the server 306 execute the care plan by combining the person's healthcare information with the client identifier to generate an order. This execution involves a machine-learning model 320 to optimize treatment recommendations based on the person's health information. The machine-learning model provides predictive insights into potential health risks, enabling proactive intervention and personalized treatment plans that significantly improve patient outcomes.

Additionally, the device 300 features an automated scheduling and reminder system driven by a sophisticated rules engine 324. This engine takes into account the patient's medical history, current health status, and standard-of-care guidelines to dynamically adjust appointment frequencies and notification schedules based on real-time data. This level of automation and personalization represents a significant advancement over traditional manual scheduling methods.

The device 300 also integrates disparate data sources, including electronic health records (EHRs), wearable devices, and patient-reported outcomes, into a cohesive and actionable format. The system's architecture includes specialized modules for data normalization, anomaly detection, and real-time alerts, which significantly enhance the ability to monitor and manage chronic conditions proactively.

In some embodiments, the processors are further configured to activate structured healthcare data received from one or more electronic health record systems to generate automated patient engagement workflows and diagnosis-driven care-management actions outside the native electronic health record environment.

Finally, the device 300 ensures security and privacy through cryptographic processes 314 used to generate a unique client identifier. This provides a robust framework for managing sensitive healthcare data, distinguishing the system from conventional methods that may lack adequate security measures.

In some embodiments, the system may include a rules engine configured to dynamically adjust engagement actions and care-management workflows based on changes in a patient's healthcare data. For example, the rules engine may modify appointment scheduling prompts, monitoring reminders, or follow-up intervals when new healthcare information is received. Such information may include new diagnosis classifications, updated laboratory values, changes in physiological measurements, or updated provider instructions. The rules engine may evaluate one or more conditions associated with a patient record and determine whether the patient should receive modified communications or scheduling recommendations.

In some embodiments, the system may employ one or more machine learning models to analyze healthcare information associated with a patient and generate predictive insights regarding patient health status or engagement needs. The machine learning models may be trained using historical healthcare data including diagnosis classifications, vital sign measurements, laboratory values, treatment histories, or patient engagement outcomes. Based on such analysis, the system may generate recommendations, alerts, or engagement workflows designed to improve patient outcomes or healthcare compliance.

In some embodiments, the system may integrate healthcare information from multiple data sources in addition to electronic health record systems. Such sources may include wearable devices, remote physiological monitoring systems, patient-reported outcomes, laboratory systems, provider systems, or other healthcare information systems. By integrating data from multiple sources, the platform may generate more comprehensive care-management workflows and engagement actions based on a broader set of patient health indicators.

In some embodiments, the platform may generate personalized healthcare recommendations based on a patient's healthcare information. Such recommendations may be generated using diagnosis classifications, vital signs, historical treatments, care-gap determinations, or engagement history associated with the patient. Recommendations may include educational health content, healthcare services, monitoring devices, wellness programs, follow-up appointments, or other condition-related healthcare resources relevant to the patient's condition profile.

In some embodiments, the system may generate real-time alerts or notifications when certain healthcare conditions or engagement thresholds are detected. For example, alerts may be generated when physiological measurements exceed predefined thresholds, when follow-up care becomes overdue, when abnormal laboratory results are detected, or when a patient fails to respond to previously transmitted engagement communications. Such alerts may be transmitted to patients, healthcare providers, administrators, or other authorized recipients through one or more communication channels.

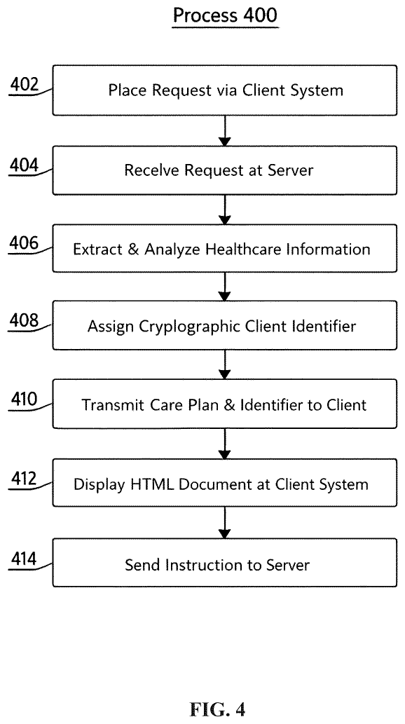

FIG. 4 is a flowchart of an example process 400. In some implementations, one or more process blocks of FIG. 4 may be performed by a device.

As illustrated in FIG. 4, process 400 may include placing a request via a client system configured to interface with a server (block 402). For example, the device may place a request via a client system configured to interface with a server, as described above. This step may include presenting a user interface on the client system for the user to input necessary information such as personal details and healthcare requirements. Additionally, it may involve validating the input data to ensure completeness and correctness before sending the request to the server.

As also illustrated in FIG. 4, process 400 may include receiving the request at the server, where the server includes a non-transitory computer-readable medium storing executable instructions (block 404). For example, the device may receive the request at the server, where the server includes a non-transitory computer-readable medium storing executable instructions, as described above. This step may involve logging the received request in a request queue and acknowledging receipt of the request to the client system. Additionally, it may include initial parsing of the request to identify key data elements.

As further illustrated in FIG. 4, process 400 may include processing the request using a specialized algorithm to extract and analyze a person's healthcare information and contact information from the client system (block 406). For example, the device may process the request using a specialized algorithm to extract and analyze a person's healthcare information and contact information from the client system, as described above. This step may include decrypting the received data if necessary and using pattern recognition techniques to identify relevant healthcare information. Additionally, it may involve storing the extracted data in a secure database for further processing.

As also illustrated in FIG. 4, process 400 may include assigning a unique client identifier to the person at the server, the identifier being generated through a cryptographic process to ensure security and privacy (block 408). For example, the device may assign a unique client identifier to the person at the server, the identifier being generated through a cryptographic process to ensure security and privacy, as described above. This step may include generating a unique identifier using a cryptographic algorithm and associating the identifier with the person's healthcare information in the database. Additionally, it may involve ensuring that the identifier does not contain any protected health information to maintain privacy.

As further illustrated in FIG. 4, process 400 may include transmitting from the server to the client system the assigned client identifier and an HTML document identifying a care plan, the document including interactive elements for user input (block 410). For example, the device may transmit from the server to the client system the assigned client identifier and an HTML document identifying a care plan, the document including interactive elements for user input, as described above. This step may involve creating an HTML document with embedded forms and interactive elements that allow the user to review and modify the care plan. Additionally, it may include securely transmitting the HTML document to the client system over a network.

As also illustrated in FIG. 4, process 400 may include receiving and storing the assigned client identifier and displaying the HTML document at the client system, the system configured to interface with user input devices (block 412). For example, the device may receive and store the assigned client identifier and display the HTML document at the client system, the system configured to interface with user input devices, as described above. This step may involve storing the client identifier in a local storage system and rendering the HTML document on the client system's user interface. Additionally, it may include configuring the client system to accept and process user inputs from various input devices such as keyboards, touchscreens, and mice.

As further illustrated in FIG. 4, process 400 may include sending to the server an instruction to process the identified care plan based on user interaction with the HTML document (block 414). For example, the device may send to the server an instruction to process the identified care plan based on user interaction with the HTML document, as described above. This step may involve capturing the user's input from the HTML document, formatting the input data into a request, and securely transmitting the request to the server. Additionally, it may include acknowledging receipt of the user's input to provide feedback to the user.

As also illustrated in FIG. 4, process 400 may include executing the care plan at the server by combining the person's healthcare information with the client identifier to generate an order, the execution involving a machine-learning model to optimize treatment recommendations based on the person's health information, where the person effects the ordering of the care plan through secure server-client communication (block 416). For example, the device may execute the care plan at the server by combining the person's healthcare information with the client identifier to generate an order, the execution involving a machine-learning model to optimize treatment recommendations based on the person's health information, where the person effects the ordering of the care plan through secure server-client communication, as described above. This step may involve retrieving the person's healthcare information using the client identifier, processing the data using a machine-learning model to generate optimized treatment recommendations, and creating an order for the care plan. Additionally, it may include securely transmitting the order to the relevant healthcare providers and confirming the execution of the care plan to the client system.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the invention as described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The preceding description of the embodiments of the present invention has been presented for illustration and description purposes. It is not intended to be exhaustive or to limit the present invention to the precise form disclosed. Many modifications and variations are possible in light of the above teaching. It is intended that the scope of the present invention be limited not by this detailed description but rather by the claims of this application. As will be understood by those familiar with the art, the present invention may be embodied in other specific forms without departing from the spirit or essential characteristics thereof. Likewise, the particular naming and division of the modules, routines, features, attributes, methodologies, and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

What is claimed is:

1. A method for managing a person's healthcare, the method comprising:
placing a request;
receiving the request at a server;
receiving a person's healthcare information at the server and contact information from a client system based on receiving the request;
assigning a client identifier to a person at the server, the client identifier absent any protected health information;
sending from the server to the client system the assigned client identifier and an HTML document identifying a care plan and including a submit button;
receiving and storing the assigned client identifier and receiving and displaying the HTML document at the client system;
sending to the server an instruction to process the identified care plan by the client system in response to a selection of the submit button; and
receiving the request and combining the person's healthcare information associated with the client identifier of the client system to generate an order to process the care plan in accordance with the person's health information, whereby the person effects the ordering of the care plan by selection of the submit button by the server.

2. The method of claim 1, wherein the person's healthcare information includes at least one of ICD-10, vital signs and other information from physician allowed access to electronic health records of a patient.

3. The method of claim 1, wherein the person's healthcare information, once received is loaded in mass and automatically processes the request, sends at least one of a text message or an email link to a patient who after entering a code, is able to open a link to view already populated data.

4. The method of claim 3, wherein the code comprises a 6-digit code.

5. The method of claim 3, further comprising receiving inputs from a user to change a patient data and an input to cause processing of the patient data.

6. A method for managing a person's healthcare, the method comprising:
placing a request via a client system configured to interface with a server;
receiving the request at the server, wherein the server includes a non-transitory computer-readable medium storing executable instructions;
processing the request using a specialized algorithm to extract and analyze a person's healthcare information and contact information from the client system;
assigning a unique client identifier to the person at the server, the identifier being generated through a cryptographic process to ensure security and privacy;
transmitting from the server to the client system the assigned client identifier and an HTML document identifying a care plan, the document including interactive elements for user input;
receiving and storing the assigned client identifier and displaying the HTML document at the client system, the system configured to interface with user input devices;
sending to the server an instruction to process the identified care plan based on user interaction with the HTML document; and
executing the care plan at the server by combining the person's healthcare information with the client identifier to generate an order, the execution involving a machine-learning model to optimize treatment recommendations based on the person's health information, whereby the person effects the ordering of the care plan through secure server-client communication.

7. The method of claim 6, wherein the person's healthcare information includes at least one of ICD-10 codes, vital signs, and other information from a physician allowed access to electronic health records of a patient.

8. The method of claim 6, wherein the person's healthcare information, once received, is loaded in mass and automatically processes the request, sending at least one of a text message or an email link to a patient who, after entering a code, is able to open a link to view already populated data.

9. The method of claim 8, wherein the code comprises a 6-digit code.

10. The method of claim 8, further comprising receiving inputs from a user to change a patient's data and an input to cause processing of the patient's data.

11. The method of claim 6, further comprising dynamically adjusting appointment frequencies and notification schedules based on real-time healthcare data using a sophisticated rules engine.

12. The method of claim 6, further comprising utilizing a machine learning model to provide predictive insights into potential health risks based on the analyzed healthcare information.

13. The method of claim 6, further comprising integrating disparate data sources, including electronic health records (EHRs), wearable devices, and patient-reported outcomes, into a cohesive and actionable format.

14. The method of claim 6, wherein the client system includes an intuitive user interface that allows patients and healthcare providers to interact seamlessly with a healthcare management platform.

15. The method of claim 6, further comprising transmitting personalized healthcare recommendations and educational content based on patient's healthcare data and ICD-10 codes.

16. The method of claim 6, further comprising securely storing and managing healthcare data using advanced encryption and secure transmission protocols to safeguard patient information.

17. The method of claim 6, further comprising providing real-time alerts and notifications to patients and healthcare providers based on patient's health status and predefined rules.

18. A device for managing a person's healthcare comprising:
one or more processors configured to:
place a request via a client system configured to interface with a server;
receive the request at the server, wherein the server includes a non-transitory computer-readable medium storing executable instructions;
process the request using a specialized algorithm to extract and analyze a person's healthcare information and contact information from the client system;
assign a unique client identifier to the person at the server, the identifier being generated through a cryptographic process to ensure security and privacy;
transmit from the server to the client system the assigned client identifier and an HTML document identifying a care plan, the document including interactive elements for user input;
receive and store the assigned client identifier and displaying the HTML document at the client system, the system configured to interface with user input devices;
send to the server an instruction to process the identified care plan based on user interaction with the HTML document; and
execute the care plan at the server by combining the person's healthcare information with the client identifier to generate an order, the execution involving a machine-learning model to optimize treatment recommendations based on the person's health information, whereby the person effects the ordering of the care plan through secure server-client communication.

19. The device of claim 18, wherein the person's healthcare information includes at least one of ICD-10 codes, vital signs, and other information from a physician allowed access to electronic health records of a patient.

20. The device of claim 18, wherein the person's healthcare information, once received, is loaded in mass and automatically processes the request, sending at least one of a text message or an email link to a patient who, after entering a code, is able to open a link to view already populated data.
